SEQUENCE LISTING (1 sequence)

SEQ ID NO: 1 - protein, 128 aa, unidentified
MAAAVEALFVVLEREGAILPRQEGFSGVYVFFSPINFVIPPMGAVMLSLRLRVCIPPGYFGRFLALTDVNQPDVFTESYIMTPDMTEELSVVLFNHGDQFFYGHAGMAVVRLMLIRVVFPVVRQASNV

(19) United States
(12) Patent Application Publication
(10) Pub. No.: US 2026/0306551 A1
(43) Pub. Date: Oct. 8, 2026

(54) ENDOTHELIAL CELLS FOR TREATMENT OF GASTROINTESTINAL FISTULA

(71) Applicants: Angiocrine Bioscience, Inc., San Diego, CA (US); Cornell University, Ithaca, NY (US)

(72) Inventors: Paul William Finnegan, Del Mar, CA (US); Jeffrey W. Milsom, New York, NY (US)

(21) Appl. No.: 19/479,531

(22) PCT Filed: Apr. 29, 2024
(86) PCT No.: PCT/US2024/026897

§ 371 Date: Oct. 28, 2025

Related U.S. Application Data
(60) Provisional application No. 63/499,189, filed on Apr. 28, 2023.

(51) Int. Cl. A61K 35/51; A61P 1/00

(52) U.S. Cl. CPC A61K 35/51; A61P 1/00

(57) ABSTRACT

The present invention relates to the use of endothelial cells for the treatment of gastrointestinal fistula and provides compositions and methods relating thereto. In some aspects of the present invention the endothelial cells are E4ORF1+ endothelial cells, such as E4ORF1+ human umbilical vein endothelial cells.

### CROSS-REFERENCE TO RELATED APPLICATIONS

This application claims the benefit of priority of U.S. Provisional Patent Application No. 63/499,189 filed on Apr. 28, 2023, the content of which is hereby incorporated by reference in its entirety.

### SEQUENCE LISTING

The instant application contains a Sequence Listing which has been submitted electronically in XML format and is hereby incorporated by reference in its entirety. Said XML copy, created on Apr. 29, 2024, is named Angio_035_WO1_Sequence_Listing, and is 2,070 bytes in size.

### INCORPORATION BY REFERENCE

For the purposes of only those jurisdictions that permit incorporation by reference, all of the references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention.

### BACKGROUND

A gastrointestinal (GI) fistula is an abnormal connection between an epithelialized surface of the gastrointestinal system and another part of the body, often the skin, that typically has the form of a tunnel referred to as a fistula tract. An anal fistula is a specific type of GI fistula in which there is an abnormal connection between the epithelialized surface of the anal canal and, most frequently, the perianal skin or perineum. Most GI fistulae occur as a complication of abdominal surgery. However, in some cases GI fistulae evolve spontaneously and may be the result of intra-abdominal inflammation or infection. Anal fistulae most commonly arise from an anorectal abscess infection, although Crohn's Disease and radiation proctitis are other major etiologies of anal fistulae. If left untreated, GI fistulae may cause serious complications including bacterial infections and life-threatening sepsis. They also drastically decrease patients' quality of life, as the symptoms of GI fistulae include abscesses, pain, discharge of pus, blood and/or intestinal contents (sometimes in large volumes), and fecal incontinence.

The primary goal of GI fistula treatment is fistula closure and healing of the fistula wound. The current standard of care treatment is fistulotomy—a surgical procedure that involves making an incision along the whole length of the fistula tract to open it up so that it can heal as a flat scar. Other current treatments include fistulectomy, fistula curettage, glue injection, advancement flap surgery, and plug placement. However, many current fistula treatments are associated with a high risk of persistence or recurrence of the fistula, and a high risk of treatment-related fecal incontinence. In particular, in the case of surgical fistulotomy and/or fistulectomy for anal fistula repair, there is often damage to sphincter tissues during surgery leading to fecal incontinence.

Accordingly, there is a need in the art for new and improved methods for the treatment of GI fistulas, including anal fistulas. The present invention addresses this need using cell therapy.

### SUMMARY OF THE INVENTION

The present invention is based, in part, on certain studies and discoveries described in the Examples section of this patent disclosure. In particular, it has now been discovered that local administration of genetically engineered human umbilical vein endothelial cells (HUVECs) to GI fistulae is safe and associated with improved fistula repair in a variety of different clinical protocols and for at least two GI fistula types, including anal fistula. Building on these studies and discoveries, the present invention provides various new and useful methods and compositions for the treatment of GI fistulae, as summarized below and as described in additional detail in the Detailed Description, Examples, and Claims sections of this patent disclosure.

Accordingly, in some embodiments the present invention provides methods for the treatment of a gastrointestinal (“GI”) fistula in a mammalian subject in need thereof, the methods comprising administering to the subject an effective amount of a composition comprising endothelial cells (ECs).

In some embodiments the GI fistula treated using the methods of the present invention is any fistula involving any portion of the GI tract. For example, in some embodiments, the GI fistula treated using the methods of the present invention is any fistula involving any portion of the small intestine or large intestine. Similarly, in some embodiments the GI fistula treated using the methods of the present invention is any fistula involving any portion of the oral cavity, esophagus, stomach, duodenum, jejunum, ileum, colon, rectum, or anus.

In some embodiments the GI fistula treated using the methods of the present invention is an anal fistula, an anorectal fistula, an anovaginal fistula, an appendiceal fistula, a biliary fistula, a cecal fistula, a colonic fistula, a colorectal fistula, a colovesical fistula, a duodenal fistula, a duodeno-colic fistula, an enterovesical fistula, an esophageal fistula, a gallbladder fistula, a gastric fistula, a gastrocolic fistula, a gastrojejunocolic fistula, an ileal fistula, a jejunal fistula, an oral fistula, a pancreatic fistula, a rectal fistula, a rectovaginal fistula, or a salivary gland fistula. In some preferred embodiments the GI fistula is an anal fistula.

In some embodiments the GI fistula treated using the methods of the present invention is an enterocutaneous fistula. In some embodiments the GI fistula treated using the methods of the present invention is a non-healing enterocutaneous fistula. In some embodiments the GI fistula treated using the methods of the present invention is an entero-enteral fistula. In some embodiments the GI fistula treated using the methods of the present invention is a non-healing entero-enteral fistula.

In some embodiments the GI fistula treated using the methods of the present invention is a blind fistula. In some embodiments the GI fistula treated using the methods of the present invention is a complete fistula. In some embodiments the GI fistula treated using the methods of the present invention is an incomplete fistula. In some embodiments the GI fistula treated using the methods of the present invention is a simple fistula. In some embodiments the GI fistula treated using the methods of the present invention is a complex fistula. In some embodiments the GI fistula treated using the methods of the present invention is a primary fistula. In some embodiments the GI fistula treated using the methods of the present invention is a recurrent fistula.

In some embodiments the endothelial cells are umbilical vein endothelial cells (UVECs). In some embodiments the UVECs are human umbilical vein endothelial cells (HUVECs). In some embodiments the UVECs or HUVECs are E4ORF1+. In preferred embodiments the endothelial cells are E4ORF1+ HUVECs.

In some embodiments the composition comprising the ECs (e.g. E4ORF1+ HUVECs) is administered to the subject locally (e.g., to the location of a GI fistula). In some embodiments the composition comprising the ECs (e.g., E4ORF1+ HUVECs) is administered to the subject by local injection. In some embodiments the composition comprising the ECs (e.g., E4ORF1+ HUVECs) is administered to the subject by percutaneous injection (e.g., to the location of a GI fistula).

In the inventive method, the total EC dose administered to the subject may be dependent on (1) the length of the fistula tract, (2) the number of injections applied along the sides of the tract (for example, one, two, or three), (3) whether or not the internal (mucosal) opening is treated (and at which dose), (4) the specific number (dose) of ECs applied per centimeter of fistula tract, and (5) the number of fistulae to be treated in the subject.

In some embodiments, the ECs are administered to subjects with a fistula tract from about 1 cm to about 8 cm in length.

In some embodiments, the ECs are administered to the subject at a dose of from about 5×10⁶ to about 200×10⁶ total ECs per fistula tract. In some embodiments, the ECs are administered to the subject at a dose of from about 5×10⁶ to about 150×10⁶ total ECs per fistula tract. In some embodiments, the ECs are administered to the subject at a dose of from about 5×10⁶ to about 120×10⁶ total ECs per fistula tract. In some embodiments, the ECs are administered to the subject at a dose of from about 5×10⁶ to about 100×10⁶ total ECs per fistula tract. In some embodiments, the ECs are administered to the subject at a dose of from about 5×10⁶ to about 50×10⁶ total ECs per fistula tract. In some embodiments, the ECs are administered to the subject at a dose of from about 5×10⁶ to about 40×10⁶ total ECs per fistula tract. In some embodiments, the ECs are administered to the subject at a dose of from about 5×10⁶ to about 30×10⁶ total ECs per fistula tract. In some embodiments, the ECs are administered to the subject at a dose of from about 5×10⁶ to about 15×10⁶ total ECs per fistula tract. In some embodiments, the ECs are administered to the subject at a dose of from about 5×10⁶ to about 7.5×10⁶ total ECs per fistula tract. In some embodiments, the ECs are administered to the subject at a dose of from about 7.5×10⁶ to about 60×10⁶ total ECs per fistula tract. In some embodiments, the ECs are administered to the subject at a dose of from about 7.5×10⁶ to about 30×10⁶ total ECs per fistula tract. In some embodiments, the ECs are administered to the subject at a dose of from about 7.5×10⁶ to about 15×10⁶ total ECs per fistula tract. In some embodiments, the ECs are administered to the subject at a dose of from about 15×10⁶ to about 30×10⁶ total ECs per fistula tract. In some embodiments, the ECs are administered to the subject at a dose of about 7.5×10⁶ total ECs per fistula tract. In some embodiments, the ECs are administered to the subject at a dose of about 15×10⁶ total ECs per fistula tract. In some embodiments, the ECs are administered to the subject at a dose of about 30×10⁶ to about 30×10⁶ total ECs per fistula tract.

In some embodiments, the ECs are administered to the subject along one side (one half) of the fistula tract. For example, in some embodiments, the ECs are administered to the subject at a dose of from about 5×10⁶ to about 200×10⁶ total ECs along one side (one half) of the fistula. In some embodiments, the ECs are administered to the subject at a dose of from about 5×10⁶ to about 150×10⁶ total ECs along one side (one half) of the fistula. In some embodiments, the ECs are administered to the subject at a dose of from about 5×10⁶ to about 120×10⁶ total ECs along one side (one half) of the fistula. In some embodiments, the ECs are administered to the subject at a dose of from about 5×10⁶ to about 100×10⁶ total ECs along one side (one half) of the fistula. In some embodiments, the ECs are administered to the subject at a dose of from about 5×10⁶ to about 50×10⁶ total ECs along one side (one half) of the fistula. In some embodiments, the ECs are administered to the subject at a dose of from about 5×10⁶ to about 40×10⁶ total ECs along one side (one half) of the fistula. In some embodiments, the ECs are administered to the subject at a dose of from about 5×10⁶ to about 30×10⁶ total ECs along one side (one half) of the fistula. In some embodiments, the ECs are administered to the subject at a dose of from about 5×10⁶ to about 15×10⁶ total ECs along one side (one half) of the fistula. In some embodiments, the ECs are administered to the subject at a dose of from about 5×10⁶ to about 7.5×10⁶ total ECs along one side (one half) of the fistula. In some embodiments, the ECs are administered to the subject at a dose of from about 7.5×10⁶ to about 60×10⁶ total ECs along one side (one half) of the fistula. In some embodiments, the ECs are administered to the subject at a dose of from about 7.5×10⁶ to about 30×10⁶ total ECs along one side (one half) of the fistula. In some embodiments, the ECs are administered to the subject at a dose of from about 7.5×10⁶ to about 15×10⁶ total ECs along one side (one half) of the fistula. In some embodiments, the ECs are administered to the subject at a dose of from about 15×10⁶ to about 30×10⁶ total ECs along one side (one half) of the fistula. In some embodiments, the ECs are administered to the subject at a dose of about 7.5×10⁶ total ECs along one side (one half) of the fistula. In some embodiments, the ECs are administered to the subject at a dose of about 15×10⁶ total ECs along one side (one half) of the fistula. In some embodiments, the ECs are administered to the subject at a dose of about 30×10⁶ total ECs along one side (one half) of the fistula.

In some embodiments, the ECs are administered to the subject along two sides of the fistula, for example at 12 o'clock and 6 o'clock positions around the circumference of the fistula. As used herein, the term “12 o'clock” refers the anterior midline position, which may also be referred to as the cephalad position, with all other positions that are referred to using the “o'clock” terminology being positioned with reference to the 12‘o’clock position. In some of such embodiments the total dose administered can be any of the dose ranges or doses listed above but split such that a portion (such as approximately half) of the total EC dose is administered to each of two side of the fistula tract. In some embodiments, the ECs are administered to the subject along two sides of the fistula (for example at 12 o'clock and 6 o'clock positions around the circumference of the fistula) at a dose of from about 5×10⁶ to about 200×10⁶ total ECs. In some embodiments, the ECs are administered to the subject along two sides of the fistula (for example at 12 o'clock and 6 o'clock positions around the circumference of the fistula) at a dose of from about 5×10⁶ to about 150×10⁶ total ECs. In some embodiments, the ECs are administered to the subject along two sides of the fistula (for example at 12 o'clock and 6 o'clock positions around the circumference of the fistula) at a dose of from about 5×10⁶ to about 120×10⁶ total ECs. In some embodiments, the ECs are administered to the subject along two sides of the fistula (for example at 12 o'clock and 6 o'clock positions around the circumference of the fistula) at a dose of from about 5×10⁶ to about 100×10⁶ total ECs. In some embodiments, the ECs are administered to the subject along two sides of the fistula (for example at 12 o'clock and 6 o'clock positions around the circumference of the fistula) at a dose of from about 5×10⁶ to about 50×10⁶ total ECs. In some embodiments, the ECs are administered to the subject along two sides of the fistula (for example at 12 o'clock and 6 o'clock positions around the circumference of the fistula) at a dose of from about 5×10⁶ to about 40×10⁶ total ECs. In some embodiments, the ECs are administered to the subject along two sides of the fistula (for example at 12 o'clock and 6 o'clock positions around the circumference of the fistula) at a dose of from about 5×10⁶ to about 30×10⁶ total ECs. In some embodiments, the ECs are administered to the subject along two sides of the fistula (for example at 12 o'clock and 6 o'clock positions around the circumference of the fistula) at a dose of from about 5×10⁶ to about 15×10⁶ total ECs. In some embodiments, the ECs are administered to the subject along two sides of the fistula (for example at 12 o'clock and 6 o'clock positions around the circumference of the fistula) at a dose of from about 7.5×10⁶ to about 60×10⁶ total ECs. In some embodiments, the ECs are administered to the subject along two sides of the fistula (for example at 12 o'clock and 6 o'clock positions around the circumference of the fistula) at a dose of from about 7.5×10⁶ to about 30×10⁶ total ECs. In some embodiments, the ECs are administered to the subject along two sides of the fistula (for example at 12 o'clock and 6 o'clock positions around the circumference of the fistula) at a dose of from about 7.5×10⁶ to about 15×10⁶ total ECs. In some embodiments, the ECs are administered to the subject along two sides of the fistula (for example at 12 o'clock and 6 o'clock positions around the circumference of the fistula) at a dose of from about ‘5×10⁶ to about 30×10⁶ total ECs. In some embodiments, the ECs are administered to the subject at a dose of about 7.5×10⁶ total ECs along two sides of the fistula. In some embodiments, the ECs are administered to the subject at a dose of about 15×10⁶ total ECs along two sides of the fistula. In some embodiments, the ECs are administered to the subject at a dose of about 30×10⁶ total ECs along two sides of the fistula.

In some embodiments the ECs are administered to subjects along three sides of the fistula tract (for example at 12 o'clock, 4 o'clock, and 8 o'clock around the circumference of the fistula) or along 4 sides of the fistula tract (for example at 12 o'clock, 3 o'clock, 6 o'clock and 9 o'clock around the circumference of the fistula). In some such embodiments the total dose administered can be any of the dose ranges or doses listed above, but split such that a portion (such as approximately one-third) of the total EC dose is administered to each of three sides of the fistula tract, or such that a portion (such as approximately one quarter) of the total EC dose is administered to each of four sides of the fistula tract.

In some embodiments, the ECs are administered to the subject at a dose of from about 5×10⁶ to about 200×10⁶ ECs per cm of fistula length. In some embodiments, the ECs are administered to the subject at a dose of from about 5×10⁶ to about 150×10⁶ ECs per cm of fistula length. In some embodiments, the ECs are administered to the subject at a dose of from about 5×10⁶ to about 120×10⁶ ECs per cm of fistula length. In some embodiments, the ECs are administered to the subject at a dose of from about 5×10⁶ to about 100×10⁶ ECs per cm of fistula length. In some embodiments, the ECs are administered to the subject at a dose of from about 5×10⁶ to about 50×10⁶ ECs per cm of fistula length. In some embodiments, the ECs are administered to the subject at a dose of from about 5×10⁶ to about 40×10⁶ ECs per cm of fistula length. In some embodiments, the ECs are administered to the subject at a dose of from about 5×10⁶ to about 30×10⁶ ECs per cm of fistula length. In some embodiments, the ECs are administered to the subject at a dose of from about 5×10⁶ to about 15×10⁶ ECs per cm of fistula length. In some embodiments, the ECs are administered to the subject at a dose of from about 7.5×10⁶ to about 50×10⁶ ECs per cm of fistula length. In some embodiments, the ECs are administered to the subject at a dose of from about 7.5×10⁶ to about 40×10⁶ ECs per cm of fistula length. In some embodiments, the ECs are administered to the subject at a dose of from about 7.5×10⁶ to about 30×10⁶ ECs per cm of fistula length. In some embodiments, the ECs are administered to the subject at a dose of from about 7.5×10⁶ to about 15×10⁶ ECs per cm of fistula length. In some embodiments, the ECs are administered to the subject at a dose of from about 15×10⁶ to about 30×10⁶ ECs per cm of fistula length. In some embodiments, the ECs are administered to the subject at a dose of about 7.5×10⁶ ECs per cm of fistula length. In some embodiments, the ECs are administered to the subject at a dose of about 15×10⁶ ECs per cm of fistula length. In some embodiments, the ECs are administered to the subject at a dose of about 30×10⁶ ECs per cm of fistula length.

In some embodiments, the ECs are also administered to subjects at the internal opening of the fistula (i.e., the ECs are administered both to the fistula tract, as described above and elsewhere herein, and at the internal opening of the fistula). In some such embodiments the ECs are administered to subjects at a sutured internal opening of the fistula. In some embodiments the dose of ECs delivered to the internal opening is from about 5×10⁶ ECs to about 200×10⁶ ECs. In some embodiments the dose of ECs delivered to the internal opening is from about 5×10⁶ ECs to about 150×10⁶ ECs. In some embodiments the dose of ECs delivered to the internal opening is from about 5×10⁶ ECs to about 120×10⁶ ECs. In some embodiments the dose of ECs delivered to the internal opening is from about 5×10⁶ ECs to about 100×10⁶ ECs. In some embodiments the dose of ECs delivered to the internal opening is from about 5×10⁶ ECs to about 50×10⁶ ECs. In some embodiments the dose of ECs delivered to the internal opening is from about 5×10⁶ ECs to about 40×10⁶ ECs. In some embodiments the dose of ECs delivered to the internal opening is from about 5×10⁶ ECs to about 30×10⁶ ECs. In some embodiments the dose of ECs delivered to the internal opening is from about 5×10⁶ ECs to about 15×10⁶ ECs. In some embodiments the dose of ECs delivered to the internal opening is from about 7.5×10⁶ ECs to about 60×10⁶ ECs. In some embodiments the dose of ECs delivered to the internal opening is from about 7.5×10⁶ ECs to about 30×10⁶ ECs. In some embodiments the dose of ECs delivered to the internal opening is from about 7.5×10⁶ ECs to about 15×10⁶ ECs. In some embodiments the dose of ECs delivered to the internal opening is from about 15×10⁶ ECs to about 30×10⁶ ECs. In some embodiments the dose of ECs delivered to the internal opening is about 7.5×10⁶ ECs. In some embodiments the dose of ECs delivered to the internal opening is about 15×10⁶ ECs. In some embodiments the dose of ECs delivered to the internal opening is about 30×10⁶ ECs. In some embodiments the dose range or dose of ECs delivered to the internal opening (as described above) is split into two or more portions and with each portion being administered to different points around the circumference of the internal opening. For example, the total cell dose may be delivered in three portions around the circumference of the internal opening; for example, at the 12 o'clock (cephalad), 4 o'clock and 8 o'clock positions around the circumference of the internal opening. In some embodiments the ECs delivered to the internal opening are administered approximately 3 mm submucosally, for example beneath a sutured internal opening, for example by injection and/or under direct visualization (e.g. using imaging guidance).

In some embodiments the subject has more than one fistula (for example, two or three or up to ten separate fistulae) to be treated.

In some embodiments one or more fistulae of the subject will be treated once, for example by administering a composition comprising ECs (e.g., E4ORF1+ HUVECs) to the subject once. In some embodiments one or more fistulae of the subject will be treated once, for example by administering a composition comprising ECs (e.g., E4ORF1+ HUVECs) at a dose range or dose as described above, once.

In some embodiments one or more fistulae of the subject will be treated more than once, for example by administering a composition comprising ECs (e.g., E4ORF1+ HUVECs) to the subject multiple times. In some embodiments one or more fistulae of the subject will be treated more than once, for example by administering a composition comprising ECs (e.g., E4ORF1+ HUVECs) at a dose range or dose as described above to the subject multiple times. In some such embodiments the time interval between treatments/administrations may be about one week, about two weeks, about three weeks, about four weeks, about five weeks, about six weeks, about seven weeks, about eight weeks, about nine weeks, about ten weeks, about twelve weeks, about fourteen weeks, about sixteen weeks, about eighteen weeks, about twenty weeks, or more. In some embodiments, the time interval between administrations is from about 2 weeks to about 12 weeks. In some embodiments, the time interval between administrations is from about 4 weeks to about 10 weeks. In some embodiments, the time interval between administrations is from 6 weeks to about 8 weeks. In some preferred embodiments, the time interval between administrations is about six weeks. In other preferred embodiments, the time interval between administrations is about twelve weeks. In some embodiments the time interval between administrations is determined on an ad hoc basis as indicated by the status of the subject's GI fistula and/or the subject's response to the prior administration.

In some embodiments, the treatment methods of the present invention involve also performing curettage of the GI fistula prior to administration of the ECs. In some such embodiments, administration of the ECs involves distributing the ECs along the length of the curetted GI fistula.

In some embodiments, the treatment methods of the present invention involve also performing a fistulotomy procedure prior to administration of the ECs, in which case the ECs can be administered to the opened fistula tract created during the fistulotomy.

In some embodiments, the treatment methods of the present invention can be performed in the absence of fistulotomy, or in the absence of other surgical GI fistula interventions such as fistulectomy. Indeed, an important aspect of the present invention is that the methods described herein, while they can be used as an adjunct to surgical interventions, can also be used as the primary treatment modality thus avoiding surgical procedures that can damage the sphincters and lead to incontinence.

In some embodiments the compositions comprising the ECs comprise ECs (e.g., E4ORF1+ HUVECs) in a saline solution (e.g., an isotonic saline or buffered saline). In some such embodiments the compositions comprise one or more additional components suitable for administration to living subjects selected from the group consisting of buffers, salts, polysaccharides, proteins, and preservatives. In some such embodiments the compositions comprise one or more of human serum albumin (HSA), Dextran40, and DMSO.

In some such embodiments the compositions comprise up to about 1% DMSO. In some such embodiments the compositions comprise up to about 0.25% DMSO. In some such embodiments the compositions comprise about 6% Dextran 40. In some such embodiments the compositions comprise about 10% HSA. In some such embodiments the compositions comprise about 6% Dextran 40 and about 10% HSA. In some such embodiments the compositions comprise about 6% Dextran 40 and about 10% HSA and about 0.25% DMSO. In some such embodiments the compositions comprise about 6% Dextran 40 and about 10% HSA in an isotonic saline or buffered saline. In some such embodiments the compositions comprise about 6% Dextran 40 and about 10% HSA and about 0.25% DMSO in an isotonic saline or buffered saline.

In some embodiments the compositions comprising the ECs comprise ECs (e.g., E4ORF1+ HUVECs) at a concentration of from about 0.5×10⁶ cells/ml to 50×10⁶ cells/ml. In some embodiments the compositions comprising the ECs comprise ECs (e.g., E4ORF1+ HUVECs) at a concentration of from about 1.0×10⁶ cells/ml to about 25×10⁶ cells/ml. In some embodiments the compositions comprising the ECs comprise ECs (e.g., E4ORF1+ HUVECs) at a concentration of about 1×10⁶ cells/ml. In some embodiments the compositions comprising the ECs comprise ECs (e.g., E4ORF1+ HUVECs) at a concentration of about 5×10⁶ cells/ml.

These and other embodiments of the invention are described further in other sections of this patent disclosure, and this Summary of the Invention is intended to be read in conjunction with all other sections of this patent disclosure. In addition, as will be apparent to those of skill in the art, certain modifications and combinations of the various embodiments described herein fall within the scope of the present invention.

### DETAILED DESCRIPTION

The “Summary of the Invention,” “Examples,” and “Claims” sections of this patent disclosure describe many of the main embodiments of the present invention. This “Detailed Description” section provides certain additional description relating to the compositions and methods of the present invention and is intended to be read in conjunction with all other sections of this patent disclosure. Furthermore, and as will be apparent to those in the art, the different embodiments described throughout this patent disclosure can be combined in different ways, regardless of any subheadings. Such combinations of the specific embodiments described herein fall within the scope of the present invention.

### Definitions

Certain definitions and abbreviations are provided below. Other terms or phrases may be defined elsewhere in this patent disclosure or may have meanings that are clear from the context in which they are used. Unless defined otherwise herein, or unless some other meaning is clear from their use in context herein, all technical and scientific terms and abbreviations used herein have the same meaning as commonly understood by one of ordinary skill in the art to which this invention is related. For example, The Dictionary of Cell and Molecular Biology (5th ed. J. M. Lackie ed., 2013), the Oxford Dictionary of Biochemistry and Molecular Biology (2d ed. R. Cammack et al. eds., 2008), and The Concise Dictionary of Biomedicine and Molecular Biology (2d ed. P-S. Juo, 2002) can provide one of skill with general definitions of some terms used herein.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Units, prefixes, and symbols are denoted in their Systéme International de Unites (SI) accepted form. Numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10.

Wherever embodiments are described with the language “comprising,” otherwise analogous embodiments described in terms of “consisting of” and/or “consisting essentially of” are included.

As used herein, the terms “about” and “approximately,” when referring to a stated numeric value, indicate a value within plus or minus 10% of the stated numeric value. In all embodiments where a numerical value is described using the terms “about” or “approximately” the analogous embodiment where the precise numerical value is used is also contemplated. For example, for an embodiment that involves a time of about 24 hours, the analogous embodiment involving a time of 24 hours (i.e., without the “about” qualification) is also contemplated.

As used herein, cells are “positive” or “+” for a started marker when that marker is expressed to a detectable degree in the cells. For example, endothelial cells are positive for E4ORF1 (i.e., are E4ORF1+) if E4ORF1 is expressed to a detectable degree by the endothelial cells. In some situations, expression of a stated marker may be assessed in comparison to, e.g., an experimental negative control.

As used herein, the abbreviation “E4ORF” refers to an open reading frame (ORF) present within the adenoviral early 4 (E4) region of an adenovirus genome, or a polypeptide/protein encoded by that ORF (whether the gene or the protein is referred to will be clear from the context of use). As used herein, the abbreviation “E4ORF1” refers to open reading frame (ORF) 1 of the early 4 (E4) region of an adenovirus genome, or a polypeptide/protein encoded by that ORF (whether the gene or the protein is referred to will be clear from the context of use).

As used herein, the abbreviation “E4ORF1+” when used with reference to a stated cell type refers to a cell that expresses E4ORF1 to a detectable degree. E4ORF1+ cells contain a recombinant E4ORF1 nucleic acid molecule and express the E4ORF1 protein.

As used herein, the abbreviation “EC(s)” refers to an endothelial cell(s).

As used herein, the abbreviation “UVEC” refers to umbilical vein endothelial cells.

As used herein, the abbreviation “HUVEC” refers to human umbilical vein endothelial cells.

As used herein, the terms “E-CELs” and “E-CEL UVECs” refer to E4ORF1+ HUVECs or compositions comprising E4ORF1+ HUVECs. Whether the cells per se or a composition comprising the cells is referred to will be clear from the context in which the terms are used. For example, reference to E-CELs in the context of administration to a subject necessarily denotes administration of a composition comprising the E-CELs.

As used herein, the term “allogeneic” means deriving from, originating in, or being members of the same species, where the members are genetically related or genetically unrelated but genetically similar. For example, in embodiments involving administration of allogeneic ECs to a subject, the allogeneic ECs are obtained from a donor of the same species as the subject to whom the cells will be administered (i.e., the recipient). In some embodiments the allogeneic cells are obtained from a donor having a full or partial MHC/HLA match to the subject to whom the cells will be administered (i.e., the recipient)—i.e., the donor of the cells and the recipient of the cells are fully or partially MHC-matched or HLA-matched. In some embodiments, the ECs are: (a) obtained from a donor, (b) maintained and/or cultured and/or expanded ex vivo, and (c) subsequently administered into a subject of the same species as the donor.

As used herein, the term “autologous” means deriving from or originating in the same subject. For example, in embodiments involving administration of autologous ECs to a subject, the autologous cells are obtained from the subject to whom they will be administered (i.e., the donor and recipient of the ECs are the same individual). In some embodiments, ECs are: (a) obtained from a subject, (b) maintained and/or cultured and/or expanded ex vivo, and (c) subsequently administered to the same subject.

As used herein, the terms “treat,” “treating,” and “treatment” refer to achieving, to a detectable degree, an improvement in, or prevention of worsening of, one or more symptoms or sequelae of the condition being treated. For example, such terms, as used herein, include achieving, to a detectable degree (and optionally to a statistically significant degree and/or to a clinically significant degree), one or more of the following “treatment outcomes,” depending on the condition being treated: (a) healing of a GI fistula, (b) increased speed of healing of a GI fistula, (c) improvement (i.e., decrease) in, or prevention of worsening of, discharge from a GI fistula, (d) improvement in, or prevention of worsening of, an abscess in the vicinity of the fistula, (e) a reduction in the frequency or speed of relapse of a GI fistula, (f) improvement (i.e., decrease) in, or prevention of worsening of, pain associated with a GI fistula, (g) healing of a GI fistula within 6 weeks of administration of a composition comprising ECs (e.g. E4ORF1+ HUVECs), and the like. Any suitable methods or clinical tests for assessing these treatment outcomes may be used and suitable methods for assessing such treatment outcomes will be well known to physicians practicing in the art of the present invention. In some embodiments, such treatment outcomes may be assessed by comparing a symptom or sequela in a single subject before and after treatment, or by comparing a symptom or sequela in a subject treated with a method as disclosed herein to that in a different individual who is not treated or is not treated or who is treated with a placebo, or by comparing a symptom or sequela in subject treated with a method as disclosed herein to a reference value for that symptom or sequela (e.g., a reference value for that symptom or sequela typically exhibited in a healthy person of the same sex, age and weight). For each of the embodiments described herein that refer to a method of treatment, an analogous method directed to achieving any one or more of the treatment outcomes listed above, or that results in any one or more of the treatment outcomes listed above, is also provided. For example, in addition to providing methods useful for the treatment of a GI fistula in subjects in need thereof comprising administering to the subjects an effective amount of a composition comprising endothelial cells (ECs), the present invention also comprises an analogous method for the treatment of a GI fistula in subjects in need thereof comprising administering to the subjects an effective amount of a composition comprising endothelial cells (ECs) wherein the treatment results in healing of a GI fistula, or increased speed of healing of a GI fistula, or healing of a GI fistula within 6 weeks of administration of the ECs (e.g. E4ORF1+ HUVECs), or improvement (i.e., decrease) in, or prevention of worsening of, discharge from a GI fistula, and/or improvement (i.e., decrease) in, or prevention of worsening of, pain associated with a GI fistula.

As used herein the term “effective amount” refers to an amount of a stated cell type (e.g., E4ORF1+ HUVECs), or a composition comprising that stated cell type, that is sufficient to achieve the stated outcome (e.g., treatment of GI fistula) to a detectable degree, or, in some cases to a specifically stated degree. In some embodiments the effective amount is specified (e.g., in terms of a number of E4ORF1+ HUVECs per fistula tract, or per centimeter of fistula tract length). In other embodiments an appropriate “effective amount” may be determined empirically, for example using standard techniques known in the art, such as dose escalation studies, and may be determined taking into account such factors as the planned route of administration, desired frequency of administration, etc. Furthermore, an “effective amount” may be determined using studies such as those described in the Examples section of this patent disclosure.

The term “engineered” when used in relation to ECs refers to ECs cells that have been engineered by man to result in the recited phenotype (e.g., E4ORF1 expression), or to express a recited protein. The term “engineered cells” is not intended to encompass naturally occurring cells, but is, instead, intended to encompass, for example, cells that comprise a recombinant nucleic acid molecule, or cells that have otherwise been altered artificially, for example so that they express a protein that they would not otherwise express, or so that they express a protein at substantially higher levels than that observed in non-engineered ECs.

As used herein the abbreviation “HLA” refers to human leukocyte antigen.

As used herein term “subject” refers to a mammalian individual. In some embodiments the subject is a human. When the subjects are human the terms “subject” and “patient” may be used interchangeably.

As used herein, the term “GI fistula” is used consistently with its normal meaning in the art. Thus, the term “GI fistula” as used herein encompasses both enterocutaneous fistula and entero-enteral fistula involving any portion of the gastrointestinal (GI) tract (such as the oral cavity, esophagus, stomach, duodenum, jejunum, ileum, colon, rectum, or anus), including, for example, anorectal fistula, anovaginal fistula, appendiceal fistula, biliary fistula, cecal fistula, colonic fistula, colorectal fistula, colovesical fistula, duodenal fistula, duodeno-colic fistula, enterovesical fistula, esophageal fistula, gallbladder fistula, gastric fistula, gastrocolic fistula, gastrojejunocolic fistula, ileal fistula, jejunal fistula, oral fistula, pancreatic fistula, rectal fistula, rectovaginal fistula, and salivary gland fistula. Anal fistula is a specific type of GI fistula in which there is an abnormal connection between the epithelialized surface of the anal canal and, most frequently, the perianal skin or perineum. Such fistulae may also be referred to as peri-anal fistula or fistula-in-ano.

The term “GI fistula” as used herein also encompasses GI fistulae of any etiology, including GI fistulae caused by, or associated with, injury, surgery, or disease (including infectious disease and inflammatory disease). For example, the term “GI fistula” includes GI fistulae caused by, or associated with, an inflammatory bowel disease such as ulcerative colitis or Crohn's disease. The term “GI fistula” also includes GI fistula of unknown etiology (i.e., idiopathic GI fistula).

### Methods of Treatment

In some embodiments the present invention provides methods for the treatment of GI fistulae in subjects in need thereof, the methods comprising administering to the subjects an effective amount of a composition comprising endothelial cells (ECs).

In some embodiments the GI fistula treated using the methods of the present invention is an enterocutaneous fistula. In some embodiments the GI fistula treated using the methods of the present invention is a non-healing enterocutaneous fistula. In some embodiments the GI fistula treated using the methods of the present invention is an entero-enteral fistula. In some embodiments the GI fistula treated using the methods of the present invention is a non-healing entero-enteral fistula.

In some embodiments the GI fistula treated using the methods of the present invention is a fistula of the small intestine. In some embodiments the GI fistula treated using the methods of the present invention is a fistula of the large intestine.

In some embodiments the GI fistula treated using the methods of the present invention is an anal fistula, an anorectal fistula, an anovaginal fistula, an appendiceal fistula, a biliary fistula, a cecal fistula, a colonic fistula, a colorectal fistula, a colovesical fistula, a duodenal fistula, a duodeno-colic fistula, an enterovesical fistula, an esophageal fistula, a gallbladder fistula, a gastric fistula, a gastrocolic fistula, a gastrojejunocolic fistula, an ileal fistula, a jejunal fistula, an oral fistula, a pancreatic fistula, a rectal fistula, a rectovaginal fistula, or a salivary gland fistula.

In some preferred embodiments the GI fistula treated using the methods of the present invention is an enterocutaneous fistula. In some preferred embodiments the GI fistula treated using the methods of the present invention is an anal fistula.

In some embodiments the GI fistula treated using the methods of the present invention is a blind fistula. In some embodiments the GI fistula treated using the methods of the present invention is a complete fistula. In some embodiments the GI fistula treated using the methods of the present invention is an incomplete fistula. In some embodiments the GI fistula treated using the methods of the present invention is a simple fistula. In some embodiments the GI fistula treated using the methods of the present invention is a complex fistula. In some embodiments the GI fistula treated using the methods of the present invention is a primary fistula. In some embodiments the GI fistula treated using the methods of the present invention is a recurrent fistula.

In some embodiments the subjects treated using the methods of the present invention are mammalian subjects. In preferred embodiments the subjects treated using the methods of the present invention are human subjects.

In some embodiments the ECs administered in the treatment methods of the present invention are autologous ECs. In some embodiments the ECs administered in the treatment methods of the present invention are allogeneic ECs.

In some embodiments the ECs administered in the treatment methods of the present invention are umbilical vein endothelial cells (UVECs). In some embodiments the ECs administered in the treatment methods of the present invention are human umbilical vein endothelial cells (HUVECs). In some such embodiments the UVECs are E4ORF1+ UVECs. In preferred embodiments the UVECs are E4ORF1+ HUVECs.

In some embodiments, the treatment methods of the present invention involve administering ECs to the subject at a certain dose or at a certain dose range. Suitable doses and dose ranges are described in the Summary of the Invention section above.

In some embodiments, the ECs are administered to the subject such that the total volume of the EC-containing composition administered in a single administration is from about 0.5 mls to about 20 mls, such as from about 5 mls to about 15 mls.

In some embodiments, the treatment methods of the present invention involve administering ECs to the subject with a certain administration schedule. In some embodiments, the treatment methods of the present invention involve administering ECs to the subject once, i.e., in a single administration (for example a single administration of a dose of ECs as specified above). In some embodiments, the treatment methods of the present invention involve administering ECs to the subject multiple times, i.e., in multiple administrations (for example where each of the multiple administrations involves administration of a dose of ECs as specified above). For example, in some embodiments, the treatment methods of the present invention involve administering ECs to the subject twice, or three times, or four times, or more. In those embodiments that involve administering ECs to the subject multiple times, the time interval between administrations may be about one week, about two weeks, about three weeks, about four weeks, about five weeks, about six weeks, about seven weeks, about eight weeks, about nine weeks, about ten weeks, about twelve weeks, about fourteen weeks, about sixteen weeks, about eighteen weeks, about twenty weeks, or more. In some embodiments, the time interval between administrations is from about 2 weeks to about 12 weeks. In some embodiments, the time interval between administrations is from about 4 weeks to about 10 weeks. In some embodiments, the time interval between administrations is from 6 weeks to about 8 weeks. In some preferred embodiments, the time interval between administrations is about six weeks. In other preferred embodiments, the time interval between administrations is about twelve weeks. In some embodiments the time interval between administrations is determined on an ad hoc basis as indicated by the status of the subject's GI fistula and/or the subject's response to the prior administration. Other suitable administration schedules are described in the Summary of the Invention section above.

In some embodiments, the treatment methods of the present invention involve administering ECs to the subject using any suitable route of administration. In preferred embodiments, the treatment methods of the present invention involve administering ECs to the subject locally at the site of the GI fistula. In some preferred embodiments, the treatment methods of the present invention involve administering ECs to the subject by local injection at the site of GI fistula, e.g., using a needle. In some preferred embodiments, the treatment methods of the present invention involve administering ECs to the subject by local injection at the site of GI fistula using a 25-gauge needle. In some embodiments, the treatment methods of the present invention involve administering ECs to the subject under direct visualization (e.g., using imaging guidance).

In some embodiments, the administration of the ECs involves depositing the ECs along the length of the GI fistula tract. In some embodiments, the administration of the ECs involves depositing the ECs in a single injection along the length of one side/half of the GI fistula tract. In some embodiments, the administration of the ECs involves depositing the ECs in two or more separate injections along the length of two or more sides of the GI fistula tract. In some embodiments, the ECs are administered to the subject along two sides of the fistula (for example at 12 o'clock and 6 o'clock around the circumference of the fistula). In some embodiments the ECs are administered to subjects along three sides of the fistula tract (for example at 12 o'clock, 4 o'clock, and 8 o'clock around the circumference of the fistula). In some embodiments the ECs are administered to subjects along four sides of the fistula tract (for example at 12 o'clock, 3 o'clock, 6 o'clock and 9 o'clock around the circumference of the fistula). In some embodiments, the administration of the ECs involves depositing the ECs in the soft tissue external to the GI fistula tract (either along one or more sides thereof). In some embodiments, the administration of the ECs involves depositing the ECs in the soft tissue external to the GI fistula tract along the length of the GI fistula tract (either along one side or more sides thereof). In some embodiments, the administration of the ECs is performed by inserting a needle/syringe (e.g., through the skin) into the soft tissues adjacent to the fistula tract and up to the level of internal opening thereof, and injecting the EC-containing composition along the length of the fistula tract as the needle is withdrawn towards the external opening of the fistula tract, thereby depositing the ECs along the length of, and external to, the fistula tract. Such a method may be used to deposit/distribute the ECs relatively uniformly along the length of the fistula tract. This procedure can be performed on either one side or more sides of the fistula tract.

In some embodiments, the subject is treated by performing curettage of the GI fistula prior to administration of the ECs. In some of such embodiments, administration of the ECs involves distributing the ECs along the length of the curetted GI fistula. In some of such embodiments, administration of the ECs involves distributing half of the ECs along one side of the curetted GI fistula and half of the ECs along the opposite side of the curetted GI fistula.

In some embodiments the methods of treatment provided herein can be performed in conjunction with performing one or more additional medical procedures (such as surgical procedures) on the subject. For example, in some embodiments the methods of treatment provided herein are performed in conjunction with performing a fistulotomy procedure on the subject (fistulotomy is a surgical procedure that involves making an incision along the whole length of the fistula to open it up so that it can potentially heal as a flat scar, and is the standard of care treatment for anal fistulas). Similarly, in some embodiments the methods of treatment provided herein are performed in conjunction with performing fistula curettage on the subject (fistula curettage is a non-surgical intervention). And, in some embodiments the methods of treatment provided herein are performed in conjunction with performing a fistulectomy procedure on the subject (fistulectomy is a surgical intervention that fully removes the fistulous tract). In some embodiments the methods of treatment provided herein are performed in conjunction with surgically closing an opening of the GI fistula, for example using sutures. In some embodiments the methods of treatment provided herein are performed in conjunction with surgically closing the internal opening of the GI fistula.

In those embodiments that involve surgically closing the internal opening of the GI fistula, additional ECs (i.e. in addition to the ECs administered as described above) can also be administered submucosally around the surgically closed internal opening of the GI fistula.

In some embodiments the methods of treatment provided herein can be performed in conjunction with administering one or more additional agents (e.g., pharmaceutical agents, biologic agents, or cell therapy agents) to the subjects, i.e., in addition to the EC-containing compositions described herein. Additional agents that can be used in such methods include growth factors, cytokines, anti-inflammatory agents, immunosuppressive agents, anti-thymocyte globulin, mycophenylate mofetil, cyclosporin, methotrexate, cyclophosphamide, Rituxan, steroids, anti-bacterial agents, anti-viral agents, and anti-fungal agents, and other agents known in the art to be useful in the treatment of GI fistula or in the context of cell therapy or transplant therapy.

In some such embodiments the one or more additional agents is administered in the same composition as the ECs (i.e., the additional agents can be added to the compositions of the present invention). In some embodiments, the one or more additional agents is administered separately from the compositions of the present invention. For example, in some embodiments, one or more additional agents can be administered to the subjects in a different composition to that in which the ECs are provided and administered to the subjects prior to, concurrently with, or after administration of the EC-containing compositions of the present invention. In some embodiments, the one or more additional agents is administered to the subject within about 5 minutes, 10 minutes, 15 minutes, 30 minutes, 1 hour, 2 hours, 3 hours, 4 hours, 5 hours, 6 hours 12 hours, 24 hours or more before or after administration of the EC-containing compositions of the present invention.

In some embodiments, the treatment methods of the present invention lead to a detectable improvement in, or prevention of worsening of, one or more symptoms or sequelae of the GI fistula being treated. In some embodiments, the treatment methods of the present invention lead to one or more of (a) healing of a GI fistula, (b) increased speed of healing of a GI fistula, (c) improvement (i.e., decrease) in, or prevention of worsening of, discharge from a GI fistula, (d) improvement in, or prevention of worsening of, an abscess in the vicinity of the fistula, (e) a reduction in the frequency or speed of relapse of a GI fistula, and/or (f) improvement (i.e., decrease) in, or prevention of worsening of, pain associated with a GI fistula, (g) healing of a GI fistula within 6 weeks of administration of the composition comprising the ECs (e.g. E4ORF1+ HUVECs). Various methods may be used to detect and/or assess the therapeutic effects of the methods of the present invention. Such methods include visual inspection, imaging studies, biopsy studies, patient questionnaires, and the like.

### Endothelial Cells

The methods and compositions of the present invention involve endothelial cells (ECs). In some embodiments the ECs are umbilical vein endothelial cells (UVECs). UVECs are isolated from umbilical cords. The UVECs disclosed herein can be isolated from the umbilical cord using any means known to those skilled in the art.

By way of example only, UVECs can be isolated from umbilical vein tissue by a process wherein tissue is excised and mechanically broken into fragments. Enzymes may also be perfused through the major vessels of the umbilical cord from to release the endothelial cells lining the interior of the vessels and any vessels downstream thereof. Once released, the cells can be suspended and cultured in a medium supportive of endothelial cell growth. In some embodiments the cell population so isolated can be further purified, for example, by immunoselection of endothelial cells using a marker or combination of markers that is selective for endothelial cells (for example, antibody directed to CD31). The endothelial cells may be expanded in culture. In one embodiment the endothelial cells are transfected with a gene conferring a growth advantage over non-transduced cells such that transduced cells grow more rapidly and/or more stably than the non-transduced cells from which they were derived.

Additional methods for preparation of UVECs are provided in the Examples section of this patent disclosure.

In some embodiments ECs (e.g., UVECs) are genetically modified.

In preferred embodiments the ECs (e.g., UVECs) are genetically modified to express the adenovirus E4ORF1 protein, i.e., they are E4ORF1+ ECs (e.g., E4ORF1+ UVECs).

In some embodiments, the ECs are engineered to express one or more ETS family transcription factors, (i.e., they are ETS+ ECs). In some embodiments, the ECs are engineered to express the ETS family transcription factor ETV2 (i.e., they are ETV2+ ECs). In some embodiments, the ECs are E4ORF1+ ETV2+ECs.

### E4ORF1

Several of the embodiments of the present invention involve ECs that are E4ORF1+—i.e., ECs that express an E4ORF1 polypeptide. The “E4ORF1” polypeptide is encoded by open reading frame (ORF) 1 of the early 4 (E4) region of the adenovirus genome. E4ORF1+ ECs for use in accordance with the present invention typically comprise a recombinant nucleic acid molecule that contains an E4ORF1 coding sequence operatively linked to a promoter suitable for expression of the E4ORF1 coding sequence in ECs.

E4ORF1 amino acid sequences and nucleotide sequences are known in the art. Any such sequences may be used in accordance with the present invention. In some embodiments the E4ORF1 polypeptide may be from any suitable adenovirus type or strain, such as human adenovirus type 2, 3, 5, 7, 9, 11, 12, 14, 34, 35, 46, 50, or 52. In some preferred embodiments the polypeptide sequence used is from human adenovirus type 5. Amino acid sequences of such adenovirus polypeptides, and nucleic acid sequences that encode such polypeptides, are well known in the art and available in well-known publicly available databases, such as the Genbank database. For example, suitable sequences include the following: human adenovirus 9 (Genbank Accession No. CAI05991), human adenovirus 7 (Genbank Accession No. AAR89977), human adenovirus 46 (Genbank Accession No. AAX70946), human adenovirus 52 (Genbank Accession No. ABK35065), human adenovirus 34 (Genbank Accession No. AAW33508), human adenovirus 14 (Genbank Accession No. AAW33146), human adenovirus 50 (Genbank Accession No. AAW33554), human adenovirus 2 (Genbank Accession No. AP.sub.-000196), human adenovirus 12 (Genbank Accession No. AP.sub.-000141), human adenovirus 35 (Genbank Accession No. AP.sub.-000607), human adenovirus 7 (Genbank Accession No. AP.sub.-000570), human adenovirus 1 (Genbank Accession No. AP.sub.-000533), human adenovirus 11 (Genbank Accession No. AP.sub.-000474), human adenovirus 3 (Genbank Accession No. ABB 17792), and human adenovirus type 5 (Genbank accession number D12587). In one embodiment the E4ORF1 sequence used is that having NCBI accession number AZR66741.1. In one embodiment the E4ORF1 sequence used is that having NCBI accession number AP_000232.1. In one embodiment the E4ORF1 sequence used is has the amino acid sequence:

(SEQ ID NO. 1)
MAAAVEALFVVLEREGAILPRQEGFSGVYVFFSPINFVIPPMGAVMLSLR
LRVCIPPGYFGRFLALTDVNQPDVFTESYIMTPDMTEELSVVLFNHGDQF
FYGHAGMAVVRLMLIRVVFPVVRQASNV.

In some embodiments the E4ORF1 polypeptide may have amino acid sequence that is, or be encoded by a nucleic acid sequence that is, a variant, derivative, mutant, or fragment of any of the specific sequences provided herein or known in the art provided that such variants, derivatives, mutants, or fragments are, or encode, a polypeptide that has one or more of the functional properties of adenovirus E4ORF1 described herein. In some embodiments, the variants, derivatives, mutants, or fragments have about an 85% identity to the known sequence, or about an 88%, 89%, 90%, 91%, 92%, 93%, 94%, 95%, 96%, 97%, 98%, or 99% sequence identity to the known sequence. In some embodiments, a variant, derivative, mutant, or fragment of a known nucleotide sequence is used that varies in length by about 50 nucleotides, or about 45 nucleotides, or about 40 nucleotides, or about 35 nucleotides, or about 30 nucleotides, or about 28 nucleotides, 26 nucleotides, 24 nucleotides, 22 nucleotides, 20 nucleotides, 18 nucleotides, 16 nucleotides, 14 nucleotides, 12 nucleotides, 10 nucleotides, 9 nucleotides, 8 nucleotides, 7 nucleotides, 6 nucleotides, 5 nucleotides, 4 nucleotides, 3 nucleotides, 2 nucleotides, or 1 nucleotide relative to the known nucleotide sequence. In some embodiments, a variant, derivative, mutant, or fragment of a known amino sequence is used that varies in length about 50 amino acids, or about 45 amino acids, or about 40 amino acids, or about 35 amino acids, or about 30 amino acids, or about 28 amino acids, 26 amino acids, 24 amino acids, 22 amino acids, 20 amino acids, 18 amino acids, 16 amino acids, 14 amino acids, 12 amino acids, 10 amino acids, 9 amino acids, 8 amino acids, 7 amino acids, 6 amino acids, 5 amino acids, 4 amino acids, 3 amino acids, 2 amino acids, or 1 amino acid relative to the known amino acid sequence.

In some embodiments E4ORF1 sequences are used without other sequences from the adenovirus E4 region—for example not in the context of the entire E4 region or not together with other ORFs in the E4 region. However, in other embodiments E4ORF1 may be used in conjunction with one or more other ORFs from the E4 region, such as E4ORF2, E4ORF3, E4ORF4, E4ORF5 or E4ORF6/7 sequences. For example, although E4ORF1 sequences can be used in constructs (such as a viral vectors) that contain other sequences, genes, or coding regions (such as promoters, marker genes, antibiotic resistance genes, and the like), in certain embodiments, the E4ORF1 sequences are used in constructs that do not contain the entire E4 region, or that do not contain other ORFs from the entire E4 region, such as E4ORF2, E4ORF3, E4ORF4, and/or E4ORF5.

E4ORF1 encoding sequences can be present in constructs or vectors that contain various other sequences, genes, or coding regions, for example, promoters, enhancers, antibiotic resistance genes, reporter genes or expression tags (such as, for example nucleotides sequences encoding GFP), or any other nucleotide sequences or genes that might be desirable.

E4ORF1-encoding nucleic acid molecules can be under the control of one or more promoters to allow for expression. Any promoter able to drive expression of the E4ORF1 nucleic acid sequences in endothelial cells can be used. Examples of suitable promoters include, but are not limited to, the CMV, SV40, RSV, HIV-Ltr, and MML promoters. The promoter can also be a promoter from the adenovirus genome, or a variant thereof. For example, in some embodiments the promoter may be a promoter that drives expression of E4ORF1 in nature in an adenovirus genome. However, in other embodiments the promoter is not one that drives expression of E4ORF1 in nature in an adenovirus genome.

The E4ORF1-encoding sequences may comprise naturally occurring nucleotides, synthetic nucleotides, or a combination thereof. For example, in some embodiments the nucleic acid molecules of the invention can comprise RNA, such as synthetic modified RNA that is stable within cells and can be used to direct protein expression/production directly within cells. In other embodiments the E4ORF1-encoding sequences can comprise DNA. In embodiments where DNA is used, the DNA sequences may be operably linked to one or more suitable promoters and/or regulatory elements to allow (and/or facilitate, enhance, or regulate) expression within cells, and may be present in one or more suitable vectors or constructs.

The E4ORF1-encoding sequences can be introduced into UVECs using any suitable system known in the art, including, but not limited to, transfection techniques and viral-mediated transduction techniques. Transfection methods that can be used in accordance with the present invention include, but are not limited to, liposome-mediated transfection, polybrene-mediated transfection, DEAE dextran-mediated transfection, electroporation, calcium phosphate precipitation, microinjection, and micro-particle bombardment. Viral-mediated transduction methods that can be used include, but are not limited to, lentivirus-mediated transduction, adenovirus-mediated transduction, retrovirus-mediated transduction, adeno-associated virus-mediated transduction and herpesvirus-mediated transduction.

In some embodiments the E4ORF1-encoding sequences are in a vector. In some embodiments the E4ORF1-encoding sequences are in a viral vector. In some embodiments the E4ORF1-encoding sequences are in a lentiviral vector. In some embodiments the E4ORF1-encoding sequences are in an adenoviral vector. In some embodiments the E4ORF1-encoding sequences are in adeno-associated virus vector. In some embodiments the E4ORF1-encoding sequences are in a retroviral vector. In some embodiments the E4ORF1-encoding sequences are in a Moloney murine leukemia virus (MMLV) vector (a type of retroviral vector).

In some embodiments the presence of E4ORF1 coding sequences in the ECs can be confirmed and/or quantified using standard nucleic acid detection and/or quantification assays known in the art, such as PCR-based techniques (e.g., quantitative PCR) and sequencing-based techniques (e.g., quantitative next generation sequencing-based techniques). In some embodiments the presence of E4ORF1 polypeptides can be confirmed and/or quantified using standard protein detection and/or quantification assays known in the art, such as antibody-based techniques. In some embodiments the expression of functional E4ORF1 polypeptide (or an appropriate amount of functional E4ORF1 polypeptide can be confirmed and/or quantified using functional assays (e.g., in vitro or in vivo assays) for any of the functional properties of E4ORF1-expressing endothelial cells that are known in the art (such as any of those described in U.S. Pat. No. 8,465,732).

The handling, manipulation, and expression of E4ORF1 sequences in ECs may be performed using conventional techniques of molecular biology and cell biology. Such techniques are well known in the art. For example, one may refer to the teachings of Sambrook, Fritsch and Maniatis eds., “Molecular Cloning A Laboratory Manual, 2nd Ed., Cold Springs Harbor Laboratory Press, 1989); the series Methods of Enzymology (Academic Press, Inc.), or any other standard texts for guidance on suitable techniques to use in handling, manipulating, and expressing nucleotide and/or amino acid sequences. Additional aspects relevant to the handling and expression of E4ORF1 sequences in endothelial cells are described in U.S. Pat. No. 8,465,732, the contents of which are hereby incorporated by reference.

### Compositions

In some embodiments the compositions used in the methods of the present invention comprise an effective amount of ECs (e.g., E4ORF1+ HUVECs). Typically, such compositions comprise ECs in a saline solution (such as an isotonic saline, physiological saline, or buffered saline) suitable for administration to a subject. In some embodiments the compositions may comprise one or more additional excipients suitable for administration to human subjects and compatible with cell therapies, such as buffers, salts, preservatives, polysaccharides (e.g., dextrans), proteins (e.g., albumin) and the like. In some embodiments the compositions may comprise a low concentration of a cryopreservative. In some embodiments the compositions of the present invention comprise Dextran 40. In some embodiments the compositions of the present invention comprise human serum albumin (HSA). In some embodiments the compositions of the present invention comprise dimethyl sulfoxide (DMSO), for example at a concentration of up to 1%, or preferably at a concentration of up to 0.25%. In some embodiments the compositions of the present invention comprise ECs (such as E4ORF1+ HUVECs), Dextran40, HSA and DMSO in an isotonic saline. Other suitable compositions are described in the Summary of the Invention section above.

In some embodiments the compositions of the present invention comprise both E4ORF1+ and E4ORF1-negative ECs. In some embodiments at least about 75% of the ECs in the compositions are E4ORF1+. In some embodiments at least about 80% of the ECs in the compositions are E4ORF1+. In some embodiments at least about 85% of the ECs in the compositions are E4ORF1+. In some embodiments at least about 90% of the ECs in the compositions are E4ORF1+. In some embodiments at least about 95% of the ECs in the compositions are E4ORF1+. In some embodiments at least about 98% of the ECs in the compositions are E4ORF1+. In some embodiments at least about 99% of the ECs in the compositions are E4ORF1+.

Typically, the ECs (e.g., E4ORF1+ HUVECs) for use in the methods of the present invention are supplied (e.g., to the medical facility where there will be administered to subjects) in frozen form—i.e., in a frozen composition containing the specified endothelial cells (e.g., E4ORF1+ HUVECs) and a cryopreservative. The frozen compositions are thawed prior to use, for example using standard methods known in the art. In some embodiments the frozen composition may be thawed and then administered to a subject without dilution. However, in other embodiments the frozen composition is thawed and then diluted in a suitable diluent to result in the generation of a composition containing the desired or specified concentration cells and the desired or specified concentrations of excipients, for example as described above.

### Kits

In some embodiments the present invention provides a kit comprising a composition according to the present invention in sterile packaging and instructions for administering the therapeutic composition to a subject for treatment of a GI fistula. Such kits will typically include a label indicating the intended use of the contents of the kit. In this context, the term “label” includes any writing or recorded material supplied on or with the kit, or that otherwise accompanies the kit.

The invention is further described with reference to the following non-limiting Examples.

### EXAMPLES

The following examples are provided to demonstrate particular situations and settings in which this technology may be applied and are not intended to restrict the scope of the invention and the claims included in this disclosure.

### Example 1

### Preparation of E4ORF1+ HUVECs

HUVECs were isolated from the umbilical vein of an umbilical cord by enzymatic digestion with collagenase. Cells were then cultured in endothelial cell growth medium before transduction with a viral vector containing an E4ORF1 expression cassette (encoding SEQ ID NO.1). The resulting E4ORF1+ HUVEC cells are referred to throughout these Examples and the Figures referenced therein as “E-CEL UVECs or “E-CEL UVEC cells.”

For some studies E-CEL UVEC cells were transferred to bioreactors and expanded to yield the desired number of cells. Expanded E-CEL UVEC cells were cryopreserved. Prior to use, E-CEL UVEC cells were thawed, optionally further cultured/expanded, and prepared in a final composition to be administered to subjects as described in subsequent Examples.

### Example 2

### Human Clinical Trial of E4ORF1+ HUVECs for Treatment of Anal Fistula

An anal fistula is an abnormal connection between the epithelialized surface of the anal canal and, most frequently, the perianal skin or perineum. Its etiology is cryptoglandular in 90% of cases, and it almost always evolves from a spontaneously draining anorectal abscess infection. A fistula is classified into simple fistula or complex fistula based on anatomical and clinical features [6]. The complex fistula is associated with high risk of persistence or recurrence of disease and with high risk of treatment related fecal incontinence. Therefore, the treatment of complex anal fistula is more challenging, whereas simple fistula can be effectively treated with fistulotomy[7, 8], which is the standard treatment strategy for simple fistula. More complex fistula treatment is challenging because of the high risk of treatment related fecal incontinence.

Non-sphincter-preserving procedures such as fistulotomy or fistulectomy have a high success rate but can be associated with fecal incontinence in patients with complex anal fistula. Therefore, clinically, these non-sphincter-preserving procedures are a last resort for patients and physicians, and are contraindicated [9-11] for complex anal fistulas. While some sphincter-preserving procedures have been developed (such as glue injection, advancement flap, and plug placement) these have lower success rates [12, 13]. Another current treatment option is seton placement, in which a reactive suture or elastic is placed through the fistula tract and tightened at regular intervals. It slowly cuts through the tract, causing scarring, so it can prevent the wide disruption of the anal sphincter associated with fistulotomy or fistulectomy. But it is not a true sphincter-preserving procedure and not every patient can tolerate it. In summary, prior to the present invention, there was no optimal treatment for patients with complex anal fistulas. Improvement in the treatment outcomes for these patients remains an unmet medical need.

The methods of the present invention offer an opportunity to treat anal fistula in a minimally invasive manner and in outpatient setting and, importantly, while structurally preserving the sphincter.

Part A of the clinical trial was designed to test the safety and efficacy of E-CEL UVEC cells as an adjunct to fistulotomy for simple fistula repair. In Part A of the trial, 6 subjects with a simple fistula were treated with fistulotomy and injection of E-CEL UVEC cells. The top-level results of Part A of the clinical trial were as follows:

TABLE 1
Results from Part A of Clinical Trial

| | | Adverse | Wound Healed |
|---|---|---|---|
| Subject | Anal Fistula | Events | at Follow-Up |
| Ang-001 | Recurrent | No | Yes |
| Ang-002 | Recurrent | No | Yes |
| Ang-004 | Primary | No | Yes |
| Ang-006 | Recurrent | No | Yes |
| Ang-007 | Primary | No | Yes |
| Ang-008 | Primary | No | Yes |

All subjects were treated at the assigned dose for each cohort (see Table 2). Each dose level was tolerated as determined by a safety monitoring committee. No adverse events (including reactions to E-CEL UVEC cells) were observed. Preliminary assessment of efficacy (including physical examination and photo documentation) indicated accelerated healing of the treated site when E-CEL UVEC cells were administered in conjunction with fistulotomy.

While fistulotomy is the standard of care for simple perianal fistulas, it is an invasive surgical procedure that risks injuring the anal sphincter muscle. Sphincter muscle injury may lead to fecal incontinence and increases subsequent risk of further anal sphincter injury if the fistula recurs. Based on the safety observed in Part A of this clinical trial with E-CEL UVEC cells injected into the fistulotomy tract, and the early signs of clinical benefit noted (during physical examination and photo documentation) with complete healing of the fistulotomy wound occurring by week 6, part B1 was added to the clinical trial investigate the potential for E-CEL UVEC cell therapy after simple fistula curettage—without fistulotomy and, hence, without risk of inducing sphincter damage and resultant fecal incontinence. Part B2 was added to the clinical trial to investigate also incorporating an additional E-CEL UVEC injection around the internal fistula opening after suturing of the opening. The treatment protocols in part B1 and B2 of the clinical trial (both of which employ injection of E-CEL UVEC cells along the curettage tract, without fistulotomy) have the potential to accelerate the time to fistula tract healing and reduce the need for more invasive and painful fistulotomy surgery or seton drainage. Percutaneous E-CEL UVEC cell injection with fistula curettage (Part B1) is a simple outpatient procedure that will cause minimal injury to the fistula tract without disturbing the integrity of the anal sphincter muscle. The procedure performed in Part B2 of the clinical trial may also allow outpatient treatment as it is a less invasive therapy than fistulotomy with reduced risk of anal sphincter muscle injury and quicker healing time because no new surgical wound is created. Delivery of ECs along one or more side of the fistula tract will induce closure of the tract and resistance to recurrence. Concurrent delivery of ECs to the internal opening of the fistula will lead to improved healing of the mucosal epithelium leading to accelerated healing and further improvement in resistance of the tract to re-opening/recurrence.

TABLE 2
Synopsis of Key Clinical Trial Protocol Parameters

| | |
|---|---|
| Clinical Phase: | 1 B |
| Sample Size: | Part A: n = 6. Part B1: n = 3. Part B2: up to 12 subjects |
| Study Population: | Adults with perianal fistula |
| Study Design: | Open-label, single-center, single-arm, study |
| Study Duration: | 24 months (6 months enrollment and 6 months follow-up for each |
| | Part A and B) |
| Study Description: | Assessment of the initial safety and feasibility of E-CEL UVEC |
| | administration as a novel engineered cell therapy for anal |
| | fistulas. |
| | In Part A, E-CEL UVECs were administered in conjunction |
| | with fistulotomy, the standard of care treatment for anal fistulas. |
| | In Part B, E-CEL UVECs are administered as primary therapy |
| | with fistula curettage-i.e., a non-surgical approach without |
| | fistulotomy. |
| | For each subject fistulotomy or fistula curettage is followed by |
| | local tissue injection of E-CEL UVEC cells. |
| Part A: | Adult subjects with simple anal fistula with 2 or fewer fistula |
| | tracts who met all eligibility criteria were enrolled into three |
| | dose cohorts, as follows: |
| | Cohort 1: 7.5 × 10⁶ cells along ½ of the fistula tract |
| | Cohort 2: 15 × 10⁶ cells along ½ of the fistula tract |
| | Cohort 3: 30 × 10⁶ cells along the whole fistula tract |
| | Each subject's safety was assessed at 14 days after the |
| | procedure. All subjects were reassessed at 6 weeks and 24 |
| | weeks after the procedure. |
| | All subjects were treated in an outpatient setting. Subjects |
| | received intravenous medication with light sedation. |
| | Examination of the anal canal, fistula tract, and surrounding |
| | structures was performed to determine the amount of anal |
| | sphincter muscle involved. The internal opening was identified |
| | and a probe inserted and passed along the fistula tract to the |
| | external opening. Local anesthetic was applied and an incision |
| | was made over the entire length of the fistula using the probe as a |
| | guide. Open tract was curetted with standard anorectal fistula |
| | curettes. |
| | In the cohorts 1 and 2, E-CEL UVEC cells were injected into |
| | one half of the tract along the whole length. |
| | In cohort 3, E-CEL UVEC cells were injected into both sides of |
| | the tract along the whole length. |
| | The wound was gently packed with sterile gauze and dressing |
| | material. |
| Part B1: | Adult subjects with simple anal fistula who meet all eligibility |
| | criteria are enrolled concurrently. All subjects are treated in the |
| | outpatient setting. Subjects may receive intravenous or oral |
| | medication with light sedation. Examination of the anal canal, |
| | fistula tract, and surrounding structures is performed to |
| | determine the amount of anal sphincter muscle involved. The |
| | internal opening is identified and a probe is inserted and passed |
| | along the fistula tract to the external opening. The length of tract |
| | is measured in centimeters. Local anesthetic is applied and the |
| | fistula tract will be curetted with standard anorectal fistula |
| | curettes. |
| | 15 × 10⁶ E-CEL UVEC cells per centimeter is administered |
| | along the entire length of the fistula tract. |
| | The total dose injected is evenly distributed. E-CEL UVEC cells |
| | are injected along two sides (180 degrees apart from each other) |
| | of the whole length of the curetted anal fistula tract with 7.5 × |
| | 10⁶ E-CEL UVEC cells per centimeter per side of the tract, |
| | spreading cells uniformly around the tract (total of 15 × 10⁶ per |
| | cm of tract length). |
| | The skin at the external opening is punctured, penetrating to the |
| | tract close to the internal opening, and the E-CEL UVEC |
| | composition is injected along the entire tract as the needle is |
| | withdrawn, taking care to avoid entry into the fistula tract. |
| | The procedure is repeated on the opposite side of the tract. The |
| | wound is gently dressed with sterile gauze. |
| Part B2: | Subjects in Part B2 have the same eligibility criteria and |
| | undergo the same procedure as in Part B1 but with the addition |
| | of the following: |
| | The internal opening is sutured under direct visualization, using |
| | an anoscope if necessary, after the whole length of the fistula |
| | tract is curetted and irrigated with sterile saline solution. |
| | In addition to administering the 15 × 10⁶ viable E-CEL UVEC |
| | cells per centimeter of the entire length of the fistula tract, |
| | another 15 × 10⁶ viable E-CEL UVEC cell dose is injected |
| | submucosally around the sutured internal opening. |
| | One third each of this dose is injected in the 12 o'clock |
| | (cephalad aspect), one third at 4 o'clock, and on third at 8 |
| | o'clock aspects of the circumference of the opening, |
| | approximately 3 mm submucosally beneath the sutured internal |
| | opening under direct visualization-i.e., the sutured internal |
| | opening being the center of the clock face. |
| | Repeat injection procedures (up to 4 injections) may be |
| | performed with identical dosage and method of the first |
| | injection procedure. |
| Primary | Part A: |
| Objective: | To assess the safety of escalating dose of E-CEL UVEC ® |
| | cells in subjects with anal fistula at 2 weeks. |
| | Part B1 & 2: |
| | To assess safety and feasibility of E-CEL UVEC treatment of |
| | simple anal fistula with curettage-i.e., without fistulotomy. |
| Secondary | To assess the safety of E-CEL UVEC treatment in subjects with |
| Objectives: | anal fistula at 6, 12, and 24 weeks. |
| | To assess the efficacy of the E-CEL UVEC treatment in |
| | subjects with anal fistula through 24 weeks. |
| Exploratory | To assess the changes in quality of life at 24 weeks (Part A) |
| Objectives: | and 1, 3, 12, and 24 weeks (Part B) as measured by Short Form |
| | Health Survey (SF-36). To assess the histologic and other |
| | changes in the healing fistula via biopsy at 2 and 6 weeks. (Part |
| | A only) |
| Endpoints: | Incidence rate of treatment emergent adverse events |
| | The rate of subjects presenting with relapse in treated fistula |
| | The rate of treatment failure defined as continuous fistula |
| | drainage or formation of abscess at the site at 6 weeks |
| | (Part B only) |
| | Changes in SF-36 scores after treatment |
| | Histologic and other assessment of biopsied |
| | tissue/comparison of treated sides (Part A only) |
| | Time to complete healing of each side (Part A) or the |
| | whole fistula tract (Part B) |
| | Change in pain using the Visual Analog Scale (VAS) |

### Example 3

### Clinical Trial of E4ORF1+ HUVECs for Treatment of Non-Healing Enterocutaneous Fistula

A clinical trial was performed as follows to treat an adult male patient with a chronic non healing small intestine to skin anterior abdominal wall fistula with two fistula tracks and two skin openings 2-3 cm apart. The fistula releases up to 2 liters of bowel contents through the abdomen per day causing extreme quality of life deficits.

The patient was brought to the operating room and injection of E-CEL UVECs was performed under sterile conditions. The patient was given moderate sedation and placed in the supine position. Imaging systems were used to allow an image of fistula tract (obtained from preoperative CT scans) to be superimposed onto a cone beam CT (cbCT) fluoroscopic screen. The fistula site was cleared of any excess granulation tissue from the skin side.

The internal opening of the fistula was closed surgically using imaging guidance. Again using imaging guidance, E-CEL UVEC cells were injected via a syringe using a 25 g needle, first injecting 15×10⁶ viable cells at the site of the internal opening, and then injecting along the entire length of the soft tissues surrounding the fistula, circumferentially injecting the cells at a dose of 15×10⁶ viable cells/cm along the fistula tract. Following the procedure, a sterile dressing was applied to the abdomen and the patient was allowed to recover from anesthesia and sent to the recovery area to be monitored.

The patient had an excellent response to the initial treatment. The patient tolerated the first injection of the E-CEL UVEC cells into the enterocutaneous fistula tract without complication or side effects. One of the two external tracts closed following the treatment, with the other tract showing significant ingrowth of healthy granulation tissue. There was also a dramatic decrease in fistula output.

In view of the success of the initial administration, a second administration of E-CEL UVEC cells was performed 6 weeks later.

Subsequent administrations led to further decrease the volume of fistula output and progressive reduction in the size of the external opening. When treatments were temporarily halted due to administrative issues, the patient exhibited a small increase in fistula output. This increase resolved on resumption of treatment with further decrease in the size of the external opening of the fistula and fistula output. Fistula output is now less than approximately 200 mL per day; the fistula opening has reduced to approximately 1-2 mm in diameter. Further treatments are expected to result in complete closure of the fistula.

### REFERENCE LIST

1. Panes, J., et al., Expanded allogeneic adipose-derived mesenchymal stem cells (Cx601)for complex perianal fistulas in Crohn's disease: a phase 3 randomised, double-blind controlled trial. Lancet, 2016. 388(10051): p. 1281-90.
2. Candotti, F., et al., Gene therapy for adenosine deaminase-deficient severe combined immune deficiency: clinical comparison of retroviral vectors and treatment plans. Blood, 2012. 120(18): p. 3635-46.
3. Gori, J. L., et al., Endothelial Cells Promote Expansion of Long-Term Engrafting Marrow Hematopoietic Stem and Progenitor Cells in Primates. Stem Cells Transl Med, 2016.
4. Poulos, M. G., et al., Endothelial transplantation rejuvenates aged hematopoietic stem cell function. J Clin Invest, 2017. 127(11): p. 4163-4178.
5. Poulos, M. G., et al., Vascular Platform to Define Hematopoietic Stem Cell Factors and Enhance Regenerative Hematopoiesis. Stem Cell Reports, 2015. 5(5): p. 881-894.
6. Steele, S. R., et al., Practice parameters for the management of perianal abscess and fistula-in-ano. Dis Colon Rectum, 2011. 54(12): p. 1465-74.
7. Abramowitz, L., et al., The outcome of fistulotomy for anal fistula at 1 year: a prospective multicentre French study. Colorectal Dis, 2016. 18(3): p. 279-85.
8. Hall, J. F., et al., Outcomes after operations for anal fistula: results of a prospective, multicenter, regional study. Dis Colon Rectum, 2014. 57(11): p. 1304-8.
9. Rizzo, J. A., A. L. Naig, and E. K. Johnson, Anorectal abscess and fistula-in-ano: evidence-based management. Surg Clin North Am, 2010. 90(1): p. 45-68, Table of Contents.
10. Whiteford, M. H., et al., Practice parameters for the treatment of perianal abscess and fistula-in-ano (revised). Dis Colon Rectum, 2005. 48(7): p. 1337-42.
11. Cavanaugh, M., N. Hyman, and T. Osler, Fecal incontinence severity index after fistulotomy: a predictor of quality of life. Dis Colon Rectum, 2002. 45(3): p. 349-53.
12. Vogel, J. D., et al., Clinical Practice Guideline for the Management of Anorectal Abscess, Fistula-in-Ano, and Rectovaginal Fistula. Dis Colon Rectum, 2016. 59(12): p. 1117-1133.
13. Abbas, M. A., C. H. Jackson, and P. I. Haigh, Predictors of outcome for anal fistula surgery. Arch Surg, 2011. 146(9): p. 1011-6.

We claim:

1. A method of treating a GI fistula in a human subject in need thereof, the method comprising administering to a human subject with a GI fistula a composition comprising E4ORF1+ human umbilical vein endothelial cells (E4ORF1+ HUVECs), wherein: (a) from about 5×10⁶ to about 200×10⁶ total E4ORF1+ HUVECs, or (b) from about 5×10⁶ to about 120×10⁶ E4ORF1+ HUVECs per cm of fistula length, are administered to the subject by local administration at the site of the GI fistula, thereby treating the GI fistula in the subject.

2. The method of claim 1, wherein the subject is also treated by performing fistulotomy.

3. A method of treating a GI fistula in a human subject in need thereof, the method comprising administering an effective amount of a composition comprising E4ORF1+ HUVECs to a human subject with a GI fistula by local administration at the site of the GI fistula, wherein the subject is not treated by fistulotomy, thereby treating the GI fistula in the subject.

4. The method of claim 3, wherein: (a) from about 5×10⁶ to about 200×10⁶ total E4ORF1+ HUVECs, or (b) from about 5×10⁶ to about 120×10⁶ E4ORF1+ HUVECs per cm of fistula length, are administered to the subject.

5. The method of claim 4, wherein: (a) from about 7.5×10⁶ to about 30×10⁶ total E4ORF1+ HUVECs, or (b) from about 7.5×10⁶ to about 30×10⁶ E4ORF1+ HUVECs per cm of fistula length, are administered to the subject.

6. The method of claim 5, wherein: (a) about 15×10⁶ to about 30×10⁶ total E4ORF1+ HUVECs, or (b) about 15×10⁶ E4ORF1+ HUVECs per cm of fistula length, are administered to the subject.

7. The method of any of the preceding claims, wherein the administration of the E4ORF1+ HUVECs comprises depositing the E4ORF1+ HUVECs along the length of the GI fistula.

8. The method of any of the preceding claims, wherein curettage of the GI fistula is performed prior to administration of the E4ORF1+ HUVECs.

9. The method of claim 8, wherein administration of the E4ORF1+ HUVECs comprises distributing the E4ORF1+ HUVECs along the length of the curetted GI fistula.

10. The method of claim 8, wherein the administration of the E4ORF1+ HUVECs comprises distributing half of the E4ORF1+ HUVECs along one side of the curetted GI fistula and half of the E4ORF1+ HUVECs along the opposite side of the curetted GI fistula.

11. The method of any of the preceding claims, further comprising surgically closing the internal opening of the GI fistula.

12. The method of any of the preceding claims, further comprising surgically closing the internal opening of the GI fistula using sutures.

13. The method of claim 12, further comprising administering E4ORF1+ HUVECs submucosally around the surgically closed internal opening of the GI fistula.

14. The method of claim 13, further comprising administering from about 15×10⁶ to 30×10⁶ E4ORF1+ HUVECs submucosally around the surgically closed internal opening of the GI fistula.

15. The method of any of the preceding claims, comprising repeating the administration of E4ORF1+ HUVECs to the subject multiple times, wherein the time interval between administrations is from about 2 weeks to about 12 weeks.

16. The method of claim 15, wherein the time interval between administrations is from about 4 weeks to about 10 weeks.

17. The method of claim 16, wherein the time interval between administrations is from about 6 weeks to about 8 weeks.

18. The method of any one of claims 15-17, wherein the administration of the E4ORF1+ HUVECs to the subject is repeated once.

19. The method of any one of claims 15-17, wherein the administration of the E4ORF1+ HUVECs to the subject is repeated twice.

20. The method of any one of claims 15-17, wherein the administration of the E4ORF1+ HUVECs to the subject is repeated three times.

21. The method of any one of claims 15-17, wherein the administration of the E4ORF1+ HUVECs to the subject is repeated four times.

22. The method of any one of the preceding claims, where the GI fistula is an enterocutaneous fistula.

23. The method of any one of the preceding claims, where the GI fistula is an entero-enteral fistula.

24. The method of any one of the preceding claims, where the GI fistula is an anal fistula, an anorectal fistula, an anovaginal fistula, an appendiceal fistula, a biliary fistula, a cecal fistula, a colonic fistula, a colorectal fistula, a colovesical fistula, a duodenal fistula, a duodeno-colic fistula, an enterovesical fistula, an esophageal fistula, a gallbladder fistula, a gastric fistula, a gastrocolic fistula, a gastrojejunocolic fistula, an ileal fistula, a jejunal fistula, an oral fistula, a pancreatic fistula, a rectal fistula, a rectovaginal fistula, or a salivary gland fistula.

25. The method of any one of the preceding claims, where the GI fistula is an anal fistula.

26. The method of any of the preceding claims, wherein the composition comprises E4ORF1+ HUVECs and one or more excipients suitable for administration to a human subject selected from the group consisting of diluents, buffers, salts, polysaccharides, proteins, and preservatives.

27. The method of claim 26, wherein the composition comprises human serum albumin (HSA).

28. The method of claim 26 or claim 27, wherein the composition comprises Dextran40.

29. The method of any one of claims 26-28, wherein the composition comprises up to about 0.25% DMSO.

30. The method of any one of claims 26-29, wherein the composition comprises HSA, Dextran40 and up to about 0.25% DMSO.

31. The method of any of the preceding claims, wherein the composition is administered locally using a needle.

32. The method of any of the preceding claims, wherein the composition is administered locally under imaging guidance.
